(19) United States
(12) Patent Application Publication
(10) Pub. No.: US 2024/0423454 A1
(43) Pub. Date: Dec. 26, 2024

(54) CHEST TUBE INSERTION DEVICE AND METHOD FOR THORACOSCOPY

(71) Applicant: THE CATHOLIC UNIVERSITY OF KOREA INDUSTRY-ACADEMIC COOPERATION FOUNDATION, Seoul (KR)

(72) Inventors: Seung Keun YOON, Bucheon-si, Gyeonggi-do (KR); Hee Kyung KIM, Seoul (KR); Ga Young YOO, Seoul (KR)

(21) Appl. No.: 18/683,711

(22) PCT Filed: Oct. 7, 2022
(86) PCT No.: PCT/KR2022/015226

§ 371 Date: Feb. 14, 2024

(30) Foreign Application Priority Data
Aug. 22, 2022 (KR) 10-2022-0104630

(51) Int. Cl. A61B 1/00; A61B 1/04; A61M 1/00

(52) U.S. Cl. CPC A61B 1/00154; A61B 1/00009; A61B 1/042; A61M 1/84

(57) ABSTRACT

A device for inserting a chest tube for thoracoscopy comprises a photographing module that photographs an inside of a subject's body and transmits a photographed in-vivo image to an outside; a chest tube that is formed in a rigid tube shape and in which the photographing module is inserted and installed; a guide part that is coupled to the photographing module and guides the photographing module into the chest tube; a handle module that is coupled to the guide part and moves the guide part and photographing module; and a mobile device that receives data from the photographing module, and searches for and reproduces the in-vivo image photographed by the photographing module in real time.

### CROSS-REFERENCE TO RELATED APPLICATION

This application claims priority to and the benefit of Korean Patent Application No. 10-2022-0104630 filed in the Korean Intellectual Property Office on Aug. 22, 2022, the entire contents of which are incorporated herein by reference.

### TECHNICAL FIELD

The disclosure relates to a device and method for inserting a chest tube for thoracoscopy. More specifically, the disclosure relates to a device for inserting a chest tube for thoracoscopy that insets a chest tube while monitoring the inside of a pleural space through images during insertion of the chest tube, inserts the chest tube at an appropriate depth, and prevents problems of damaging organs.

### BACKGROUND ART

When a pneumothorax or pleural effusion occurs and there is a need to properly drain it, a chest tube insertion surgery is performed.

As shown in FIG. 1, in order to insert a chest tube into a pleural space, a procedure is performed through the axillary line in the center of the space between the fifth ribs of the human body to treat a patient's pneumothorax, etc. If the patient is conscious, after local anesthesia, the location of the pleural space is identified with a syringe, and then an incision is made with a surgical scalpel slightly below the location where the chest tube will enter (about 2 to 3 cm horizontally just above the lower ribs in the space between the ribs).

The process of inserting a chest tube into the pleural space of the human body according to conventional technology is described as follows. After a surgical instrument called Mosquito is pushed into the incised area of the human body to open subcutaneous tissues, muscle tissues are peeled off, and then the resulting exposed pleura is pierced to create an incision window. Then, an operator uses Long Kelly and his or her fingers to widen a penetration window in the pleura to create a space in advance for the chest tube to enter the pleural space.

Korean Registered Patent No. 10-0411226 (title of the invention: chest tube inserter) includes a head portion bent in a predetermined direction to allow a chest tube to be introduced and discharged; a neck portion connected to the head portion and supporting the introduced chest tube; a body portion extending from the neck portion; a handle module coupled to an end of the body portion; and a flow stopper that is inserted into the body portion and coupled to the end of the introduced chest tube, and flows from the handle module to the neck portion to discharge the coupled chest tube to an outside.

However, with the above device, the chest tube was inserted based on the operator's sense without viewing the inside of the chest cavity. Because the chest tube was inserted based on the operator's sense, a problem occurred in which organs such as the lungs were damaged during insertion of the chest tube.

Therefore, there is a need for a chest tube insertion device that inserts the chest tube at an appropriate depth while viewing the inside of the pleural space through images and prevents problems of damaging organs during chest tube insertion.

### DOCUMENTS OF RELATED ART

Korean Registered Patent No. 10-0411226

### DISCLOSURE

### Technical Problem

An object of the disclosure to solve the above problems is to insert a chest tube at an appropriate depth.

Additionally, an object of the disclosure is to prevent problems causing damage to organs when inserting a chest tube.

In addition, an object of the disclosure is to effectively remove drainage substances by placing a chest tube in a more accurate position through a photographing module.

In addition, an object of the disclosure is to perform a procedure while an operator identifies images, regardless of location.

The technical objects to be achieved by the disclosure are not limited to the technical objects mentioned above, and other technical objects not mentioned may be clearly understood by those skilled in the art from the following descriptions.

### Technical Solution

In order to achieve the above object, the configuration of the disclosure may comprise a photographing module that photographs an inside of a subject's body and transmits a photographed in-vivo image to an outside; a chest tube that is formed in a rigid tube shape and in which the photographing module is inserted and installed; a guide part that is coupled to the photographing module and guides the photographing module into the chest tube; a handle module that is coupled to the guide part and moves the guide part and photographing module; and a mobile device that receives data from the photographing module, and searches for and reproduces the in-vivo image photographed by the photographing module in real time.

In one embodiment of the disclosure, the photographing module and guide part may be selectively separated from the chest tube.

In one embodiment of the disclosure, the photographing module may include a photographing part that photographs the inside of the subject's body; and a transmitting part that is coupled to the photographing part and receives the in-vivo image transmitted from the photographing part to transmit the in-vivo image to the mobile device through the wired or wireless communication method.

In one embodiment of the disclosure, the photographing part may include a camera that photographs the inside of the subject's body; and a lighting device that is coupled with the camera, formed in plural number, and emits light to illuminate the inside of the body.

In one embodiment of the disclosure, the photographing module may include a camera cover that is in contact with the photographing module and is formed in a shape surrounding the photographing module; and a pin part that is provided with a plurality of pins formed in an upward direction from an outer surface of the camera cover and is in contact with the chest tube.

In one embodiment of the disclosure, a material of the camera cover may be formed of synthetic resin, rubber, and silicone.

In one embodiment of the disclosure, the plurality of pins may be inclined to form an acute angle with a flow direction of fluid flowing in one direction.

In one embodiment of the disclosure, in the plurality of pins, a pin angle α forming an acute angle may be formed between a tangent line parallel to a central axis of the camera cover while contacting the plurality of pins on an outer peripheral surface of the camera cover and the plurality of pins.

In one embodiment of the disclosure, when an external force is applied to the plurality of pins, the pin angle is changed by the external force, so that an inner surface of the chest tube comes into contact with the plurality of pins, and the plurality of pins and the chest tube may be fixed.

In one embodiment of the disclosure, the pin part is formed in plural number, and fluid may flow along a space formed by the camera cover and the plurality of pin parts.

In one embodiment of the disclosure, a method for inserting a chest tube for thoracoscopy may comprise the steps of (a) cutting an area to be operated and making a hole in a pleura; (b) combining the photographing module and camera fixation module and inserting the photographing module and camera fixation module in the pleura space, (c) photographing the inside of the subject's body by the photographing module when the device for inserting the chest tube is inserted into the subject's pleural space, and then transmitting the photographed in-vivo image to the mobile device using the wired or wireless communication method, (e) inserting the chest tube based on the in-vivo image reproduced on the mobile device, and (f) connecting the chest tube to the drainage container.

In one embodiment of the disclosure, the drainage container may store drainage substance located in the subject's pleural space.

### Advantageous Effects

The effect of the disclosure according to the above configuration has the advantage of facilitating drainage by inserting the chest tube at an appropriate depth.

In addition, the effect of the disclosure has the advantage of preventing damage to organs when inserting a chest tube by monitoring the body through images.

In addition, the effect of the disclosure has the advantage of being able to use currently employed surgical methods and surgical tools as is by adding the present device to the conventionally employed chest tube.

In addition, the effect of the disclosure is that a degree of drainage of the drainage substance can be monitored without separating the chest tube and photographing module. Thus, when the drainage substance occurs in multiple locations, the pleural space is monitored with the photographing module and the chest tube is moved to easily remove the drainage substance, and when drainage is completed and the chest tube is removed from the pleural space, the insider of the body can be monitored with the photographing module to prevent the problem of damaging organs.

In addition, the effect of the disclosure has the advantage that the operator can perform the procedure while identifying the image, regardless of location.

The effects of the disclosure are not limited to the above-mentioned effects, and it should be understood that the effects of the disclosure include all effects that could be inferred from the configuration of the invention described in the detailed description of the invention or the appended claims.

### DESCRIPTION OF DRAWINGS

FIG. 1 is a diagram illustrating a method for inserting a chest tube according to an embodiment of the disclosure.

FIG. 2 is a conceptual diagram illustrating a device for inserting a chest tube that transmits photographed images to a mobile device through a wired or wireless communication method according to an embodiment of the disclosure.

FIG. 3 is a diagram illustrating separation of a photographing module and chest tube of a device for inserting a chest tube according to an embodiment of the disclosure.

FIG. 4 is a diagram illustrating insertion of a display device into a chest tube according to an embodiment of the disclosure.

FIG. 5 is a conceptual diagram illustrating a device for inserting a chest tube that transmits photographed images to a mobile device through a wired or wireless communication method according to an embodiment of the disclosure.

FIG. 6 is a front view, plan view, and combined view of a chest tube fixation module according to an embodiment of the disclosure.

FIG. 7 is a diagram illustrating an angle of a chest tube fixation module according to a diameter of a chest tube according to an embodiment of the disclosure.

FIG. 8 is a diagram illustrating a chest tube fixation module coupled with a guide part according to an embodiment of the disclosure.

FIG. 9 is a conceptual diagram illustrating a device for inserting a chest tube that transmits photographed images to a mobile device through a wired or wireless communication method according to an embodiment of the disclosure.

FIG. 10 is a front view and top view of a camera fixation module according to an embodiment of the disclosure.

FIG. 11 is a diagram illustrating an angle of a camera fixation module according to a diameter of a chest tube according to an embodiment of the disclosure.

FIG. 12 is a diagram illustrating a camera fixation module coupled with a guide part according to an embodiment of the disclosure.

### BEST MODE FOR INVENTION

The most preferred embodiment according to the disclosure comprises a photographing module that photographs an inside of a subject's body and transmits a photographed in-vivo image to an outside; a chest tube that is formed in a rigid tube shape and in which the photographing module is inserted and installed; a guide part that is coupled to the photographing module and guides the photographing module into the chest tube; a handle module that is coupled to the guide part and moves the guide part and photographing module; and a mobile device that receives data from the photographing module, and searches for and reproduces the in-vivo image photographed by the photographing module in real time, wherein when the chest tube coupled with the photographing module is inserted into a pleural space, the photographing module photographs an inside of the pleural space and transmits the photographed image to the mobile device through a wired or wireless communication method, so that an operator monitors the inside of the pleural space regardless of location.

### Mode for Invention

Hereinafter, the disclosure will be explained with reference to the accompanying drawings. The disclosure, however, may be modified in various different ways, and should not be construed as limited to the embodiments set forth herein. Also, in order to clearly explain the disclosure, portions that are not related to the disclosure are omitted, and like reference numerals are used to refer to like elements throughout the specification.

In the whole specification, it will be understood that when an element is referred to as being “connected (joined, contacted, or coupled)” to another element, it can be “directly connected” to the other element or it can be “indirectly connected” to the other element with other elements being interposed therebetween. In addition, it will be understood that when a component is referred to as “comprising or including” any component, it does not exclude other components, but can further comprise or include the other components unless otherwise specified.

The terminology used in the specification is for the purpose of describing particular embodiments only and is not intended to limit the disclosure. The singular forms are intended to include the plural forms as well, unless context clearly indicates otherwise. It will be further understood that the terms “comprises/includes” or “comprising/including” when used in this specification, specify the presence of stated features, integers, steps, operations, elements, components, or combinations thereof, but do not preclude the presence or addition of one or more other features, integers, steps, operations, elements, components, and combinations thereof.

Hereinafter, the disclosure will be described in detail with reference to the accompanying drawings.

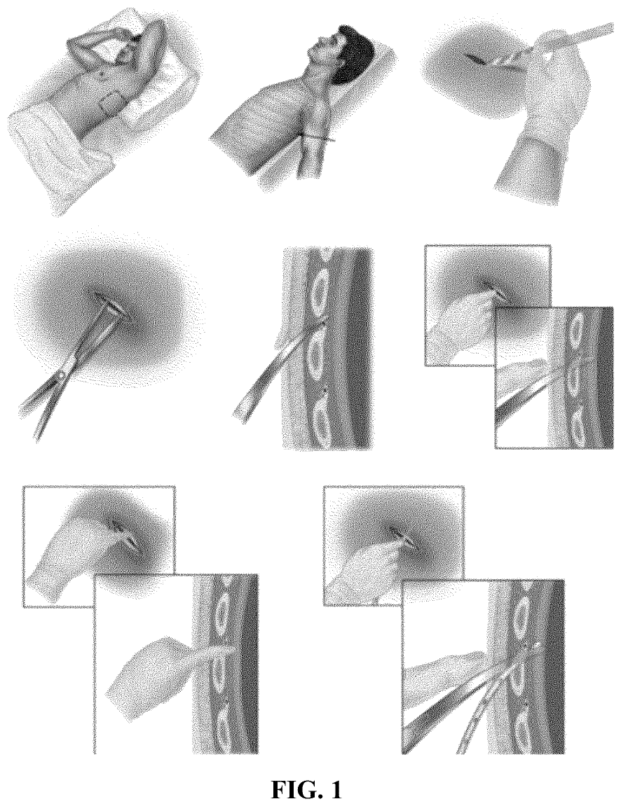

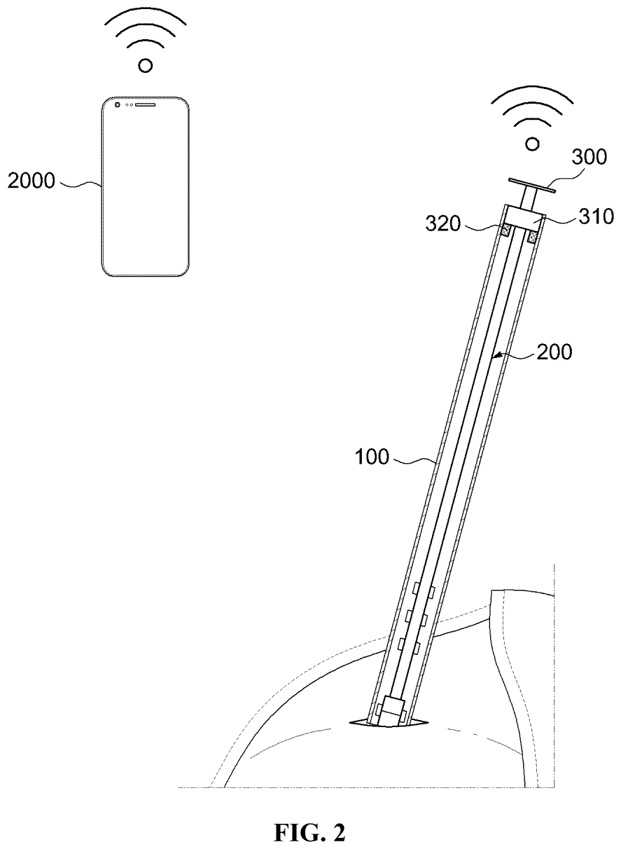

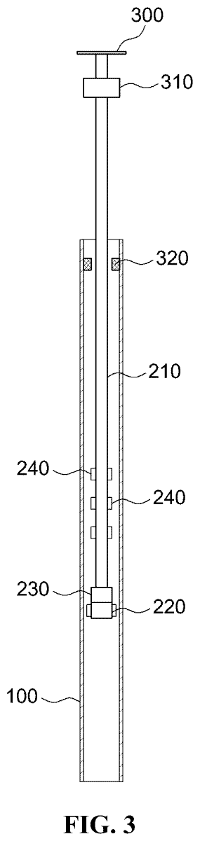

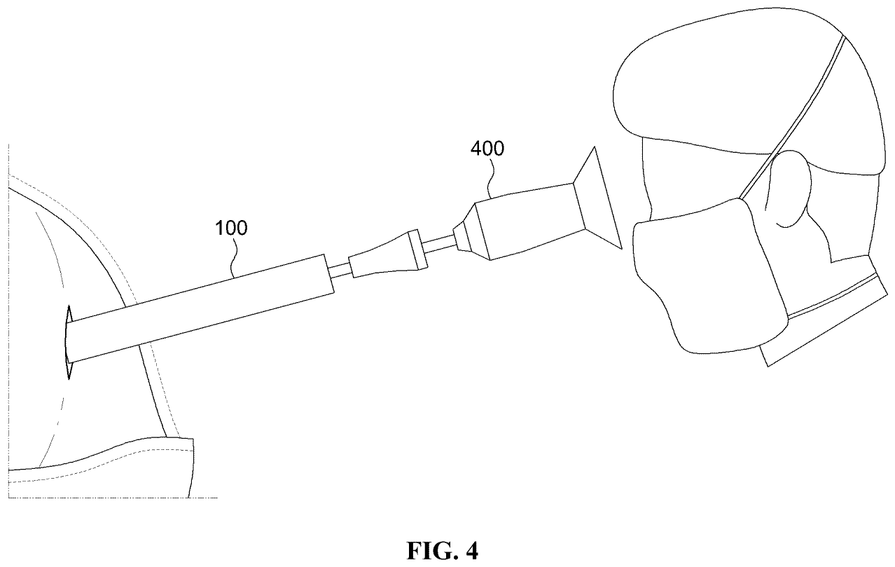

FIG. 1 is a diagram illustrating a method for inserting a chest tube 100 according to an embodiment of the disclosure, FIG. 2 is a conceptual diagram illustrating a device for inserting a chest tube 100 that transmits photographed images to a mobile device 2000 through a wired or wireless communication method according to an embodiment of the disclosure, FIG. 3 is a diagram illustrating separation of a photographing module 200 and chest tube 100 of a device for inserting a chest tube 100 according to an embodiment of the disclosure, and FIG. 4 is a diagram illustrating insertion of a monitoring device 400 into a device for inserting a chest tube 100 according to an embodiment of the disclosure.

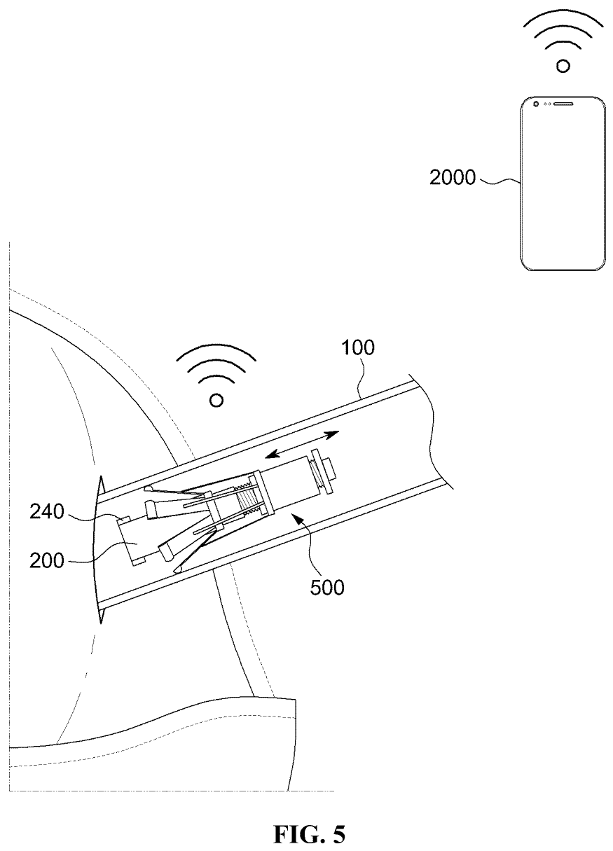

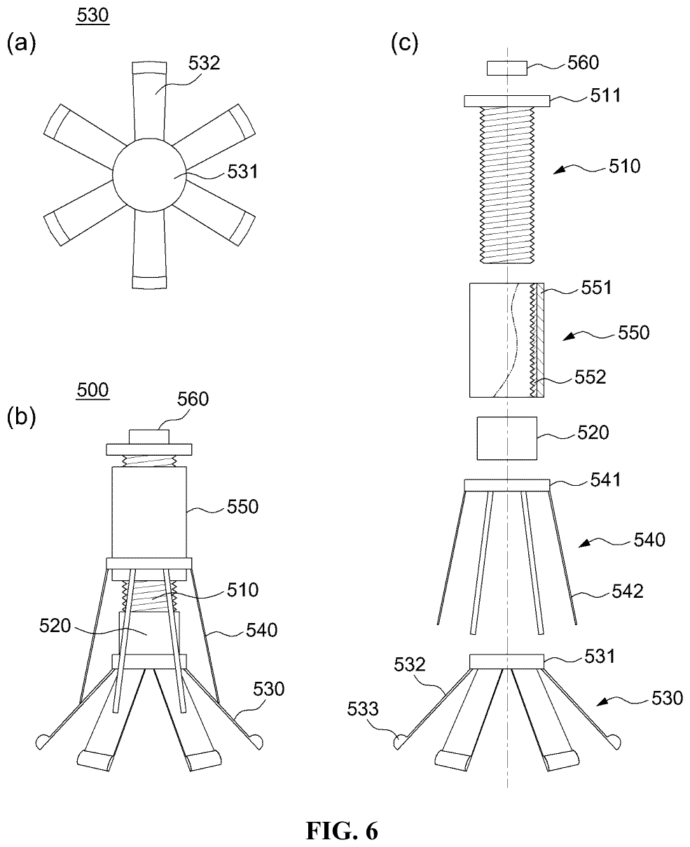

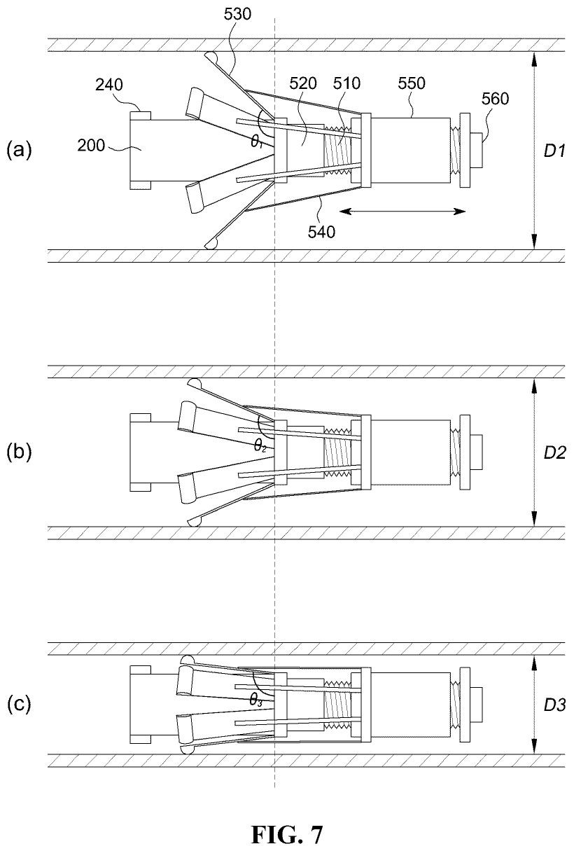

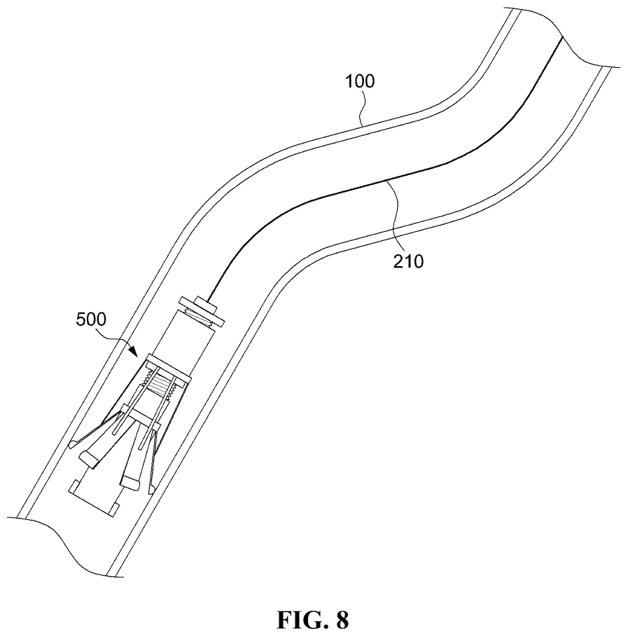

FIG. 5 is a conceptual diagram illustrating a device for inserting a chest tube that transmits photographed images to a mobile device through a wired or wireless communication method according to an embodiment of the disclosure, FIG. 6 is a front view, plan view, and combined view of a chest tube fixation module according to an embodiment of the disclosure, FIG. 7 is a diagram illustrating an angle of a chest tube fixation module according to a diameter of a chest tube according to an embodiment of the disclosure, and FIG. 8 is a diagram illustrating a chest tube fixation module coupled with a joint part according to an embodiment of the disclosure.

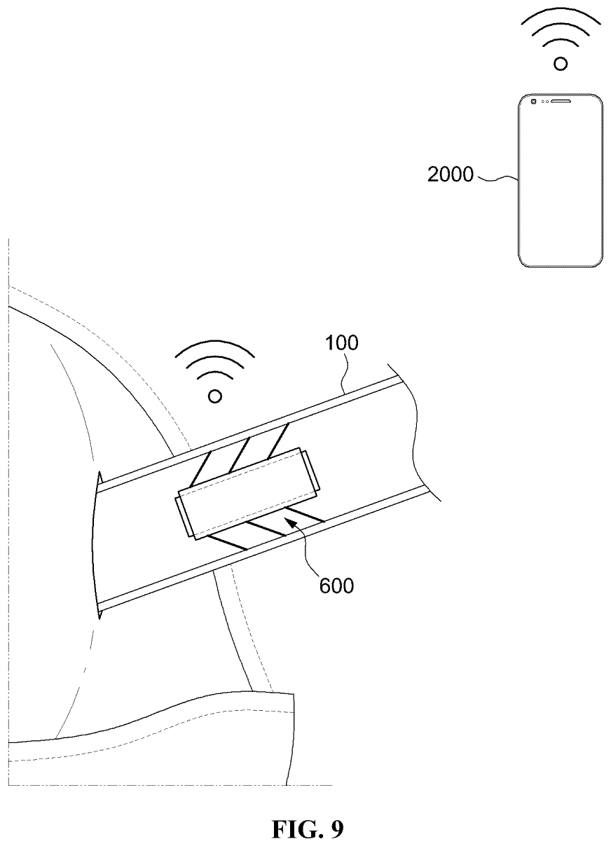

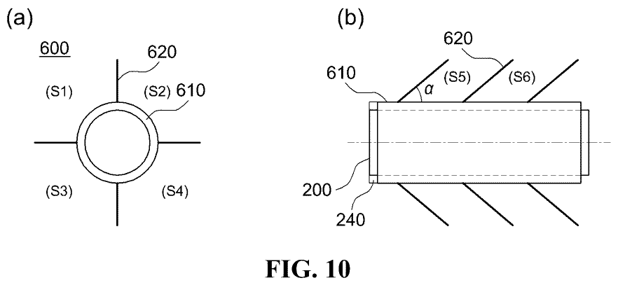

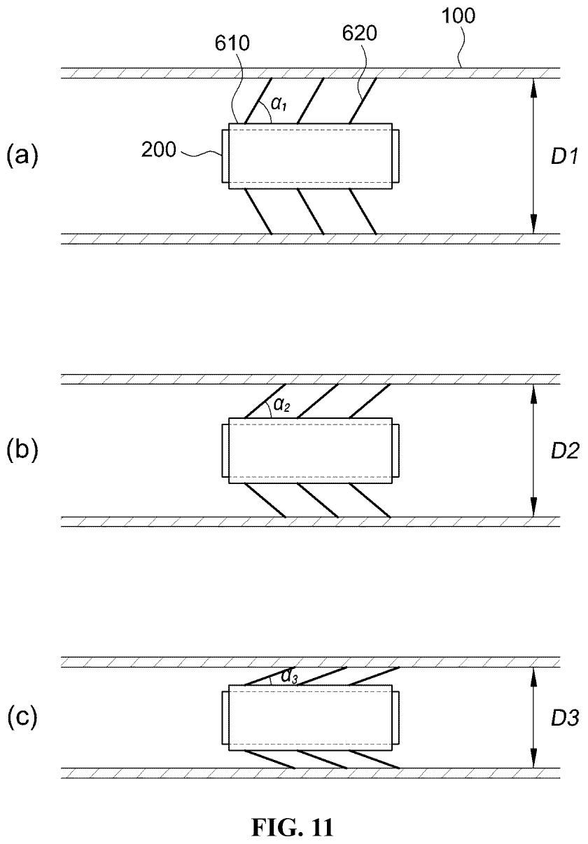

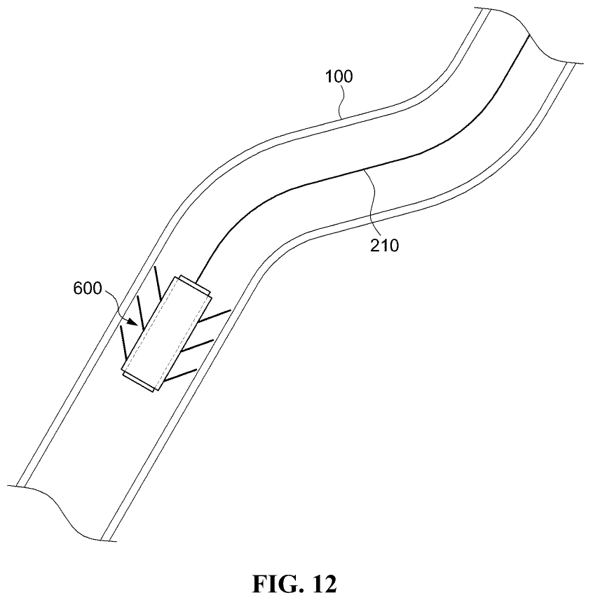

FIG. 9 is a conceptual diagram illustrating a device for inserting a chest tube that transmits photographed images to a mobile device through a wired wireless communication method according to an embodiment of the disclosure, FIG. 10 is a front view and top view of a camera fixation module according to an embodiment of the disclosure, FIG. 11 is a diagram illustrating an angle of a camera fixation module according to a diameter of a chest tube according to an embodiment of the disclosure, and FIG. 12 is a diagram illustrating a camera fixation module coupled with a joint part according to an embodiment of the disclosure.

As shown in FIGS. 2 and 3, a device for inserting a chest tube 100 for thoracoscopy may comprise a photographing module 200 that photographs an inside of a subject's body and transmits a photographed in-vivo image to an outside, a chest tube 100 that is formed in a rigid tube shape and in which the photographing module 200 is inserted and installed, a guide part 210 that is coupled to the photographing module 200 and guides the photographing module 200 into the chest tube 100, a handle module 300 that is coupled to the guide part 210 and moves the guide part 210 and photographing module 200, and a mobile device 2000 that is connected to the photographing module 200 and searches for and reproduces the in-vivo image photographed by the photographing module 200 in real time.

The chest tube 100 may be formed in a rigid and transparent tubular shape. The photographing module 200 may be inserted into one side of the chest tube 100, and the other side of the chest tube 100 may be coupled with a drainage container to drain substances located in the subject's pleural space into the chest tube 100. The chest tube 100 is introduced into the drainage container, so that the drainage fluid located in the pleural space moves along the chest tube 100 and may be introduced into the drainage container.

The material of the chest tube 100 may be made of various materials generally used in medical tubes, and may preferably be made of one or more materials selected from the group consisting of glass, elastomer, silicone, and soft resin, but is not limited thereto.

The material of the chest tube may be formed of, for example, polymerized material such as Nylon, Dacron, synthetic polyamide, expanded polytetrafluro-ethylene, polyethylene and ultra-high molecular weight fibers of polyethylene, or stainless steel, cobalt-chromium alloy, titanium, titanium alloy or nickel-titanium shape memory alloys, or other materials.

The device for inserting the chest tube 100 for thoracoscopy is coupled with the photographing module 200 and chest tube 100 and may further include the handle module 300 for moving the photographing module 200.

Here, the chest tube 100 may be formed in the shape of a transparent tube made of a rigid material in order to have strength to withstand the external force generated when the chest tube 100 is inserted into the patient's body. The photographing module 200 may be inserted into the chest tube 100, and the chest tube 100 may be selectively coupled with a chest tube fixture 320, which is coupled with the handle module 300, and light from a lighting device 240 may be projected into the body through the transparent chest tube 100.

The chest tube 100 may be provided with a fixing hole formed in the shape of a hole from the lower edge toward an upper direction, and a plurality of fixing holes may be formed. A protruding device formed on the chest tube fixture 320 may be inserted into the fixing holes formed on the chest tube 100. The handle module 300 is in contact with the chest tube fixture 320, so that the chest tube 100 and the handle module 300 may be fixed.

As shown in FIG. 3, the photographing module 200 coupled with the handle module 300 is inserted in a direction from the other side to one side of the chest tube 100 until the chest tube fixture 320 and the handle fixture 310 of the handle module 300 contact, so that the chest tube 100 and the handle module 300 can be coupled, and the photographing module 200 can be inserted into the chest tube 100 to easily observe the pleural space.

In this case, the inner diameter of the chest tube fixture 320 may be formed larger than the photographing module 200 and guide portion 210, and as a result, the photographing module and guide part can be introduced and removed through the inner diameter of the chest tube fixture.

The chest tube 100 may be separated from the handle module 300 by moving the photographing module 200 coupled with the handle module 300 from one side to the other side of the chest tube 100. As a result, the chest tube 100, which is separated from the handle module 300, may be coupled with a drainage container to drain substances located in the subject's pleural space into the chest tube 100. The chest tube 100 is introduced into the drainage container, so that the drainage fluid located in the pleural space moves along the chest tube 100 and may be introduced into the drainage container.

In another embodiment, when separating the second chest tube fixture 320 from the chest tube 100, the photographing module 200 located inside the chest tube 100 may be removed. As a result, the chest tube 100 may be coupled with a drainage container to drain substances located in the subject's chest tube into the inside of the chest tube 100. The chest tube 100 is introduced into the drainage container, so that the drainage fluid located in the pleural space moves along the chest tube 100 and may be introduced into the drainage container.

The photographing module 200 is characterized by photographing the inside of the subject's body, and may include a photographing part 220, and a transmitting part 230 that is connected to the photographing part 220 and transmits data photographed by the photographing part 220 to the outside.

The photographing part 220 may include a camera that photographs the inside of the subject's body and transmits a photographed in-vivo image to the outside using a wired or wireless communication method, the lighting device 240 that is coupled with the camera and emits light, and a battery that supplies power.

The camera may include a lens adjacent to an image sensor, which may be a CMOS or CCD device, and may be constructed including a kit and cover glass. The camera may photograph organs inside the body that are reflected in the lighting device 240, and transmit the photographed interior of the body to the outside through the transmitting part 230.

A plurality of lighting devices 240 may be provided, and the lighting devices 240 may be provided symmetrically on the left and right sides adjacent to the camera, so that the plurality of lighting devices 240 may be formed to emit light to illuminate the body.

The plurality of lighting devices 240 may be provided on the guide part 210 at a predetermined distance from the camera, and the light of the lighting devices 240 may be projected into the body by the transparent chest tube 100. In this case, due to the chest tube 100 formed in a transparent shape, the plurality of lighting devices 240 mounted on the guide part 210 can illuminate the body and increase luminous power, allowing the camera to obtain clearer image quality when photographing the body. This allows the chest tube to be inserted at an appropriate depth and the chest tube 100 to be accurately moved to the location where the drainage substance is located.

The photographing part 220 may be provided with the battery that supplies power to the photographing part 220 and transmitting part 230, and power may be transmitted from the battery to supply power to the photographing part 220 and the transmitting part 230. In another embodiment, a power supply part may be connected to the photographing part 220 and transmitting part 230 by a wire, and may be connected to an outlet to supply power to the photographing part 220 and transmitting part 230.

The transmitting part 230 may receive the in-vivo image transmitted from the photographing part 220 and transmit it to the mobile device 2000 through wired or wireless communication methods. The in-vivo image photographed by the photographing part 220 is connected to the mobile device 2000 through a wire, so that the operator can perform a procedure while identifying the image regardless of location. In another embodiment, the in-vivo image may be transmitted to the mobile device 2000 wirelessly through a mobile communication network or wireless Internet network.

By observing the in-vivo image transmitted to the mobile device 2000 through the display formed on the mobile device 2000, the chest tube 100 can be inserted at an appropriate depth to facilitate drainage, problems causing damage to organs can be prevented when inserting the chest tube 100, and the operator can proceed with the procedure while identifying the images, regardless of location.

One side of the guide part 210 can be coupled with the photographing part 220 and transmitting part 230, and one side of the guide part 210 has threads and can be screwed together with the photographing part 220. Accordingly, the photographing part 220 and the guide part 210 can be separated and easily cleaned. Additionally, by inserting a connection line inside the guide part, it can be wiredly connected to the photographing part 220 and transmitting part 230.

The material of the guide part 210 may be made of one or more materials selected from surgical metal and surgical plastic.

In another embodiment, as shown in FIG. 2, one side of the guide part 210 may be coupled with the photographing part 220 and transmitting part 230, and the other side of the guide part 210 may be coupled with the handle module 300. One side and the other side of the guide part 210 have threads and can be screwed to the handle module 300 and photographing part 220.

Because of this, the guide part 210, photographing part 220, and handle module 300 can be separated and easily cleaned.

As shown in FIG. 2, the handle module 300 is formed in a shape that the operator can grasp, and the operator may move the photographing module 200 and chest tube 100 by grasping the handle module 300. The handle module 300 has a handle plate that is formed in a plate shape, and has a handle bar that extends upward from the center of the handle plate, so that it can be formed in a ‘ [symbol] ’ shape. One side of the handle bar may be in contact with one finger, and the other side of the handle bar may be in contact with another finger, so that the handle module 300 can be easily moved. The handle bar may be threaded downward from the upper edge and be screwed to the handle fixture 310.

The handle fixture 310 may be formed in a cylindrical shape, and the diameter of the handle fixture 310 may be formed to be the same as the diameter of the inner surface of the chest tube 100, and the handle fixture 310 may be replaced according to the diameter of the chest tube 100. Due to this, the outer surface of the handle fixture 310 is in contact with the inner surface of the chest tube 100, so that the photographing module 200 is easily inserted and fixed inside the chest tube 100, and thus, the chest tube 100 is inserted at an appropriate depth when inserting the chest tube 100 and problems of damaging organs can be prevented.

A handle fixing hole formed in the shape of a hole may be provided in the center of one side of the handle fixture 310, and a thread is formed in the handle fixing hole so that one side of the handle fixture 310 and the handle bar of the handle module 300 can be screwed together.

An endoscope fixing hole formed in the shape of a hole may be provided in the center of the other side of the handle fixture 310, and a thread is formed in the endoscope fixing hole so that the other side of the guide part 210 of the photographing module 200 and the other side of the handle fixture 310 can be screwed together.

The handle fixture 310 can be inserted into the chest tube 100, and can fix the chest tube 100 and the handle module 300 by contacting the chest tube fixture 320.

The chest tube fixture 320 is coupled to the inner surface of the chest tube 100 and contacts the handle fixture 310 to prevent the movement of the handle module 300, thereby fixing the handle module 300 to the chest tube 100. When the photographing module 200 moves in a direction from the other side to one side of the chest tube 100, the chest tube fixture 320 and the handle fixture 310 come into contact, so that the chest tube 100 can be fixed to the handle fixture module.

The chest tube fixture 320 may be formed in a circular plate shape, and the diameter of the chest tube fixture 320 may be formed to be the same as the diameter of the inner surface of the chest tube 100. The chest tube fixture 320 may be provided with an endoscope hole formed in the shape of a hole at the inner center of the chest tube fixture 320.

The diameter of the endoscope hole formed in the chest tube fixture 320 may be larger than the diameters of the photographing part 220, transmitting part 230, and guide part 210. Because of this, the photographing module 200 may be easily separated from the chest tube 100 or the photographing module 200 may be inserted into the chest tube 100.

The chest tube fixture 320 may be provided with a protruding device formed in a protruding shape on the outer surface of the chest tube fixture 320. The protruding device may move in a downward direction when an external force is applied, and may be restored to its original shape and have a protruding shape when the external force is removed.

Accordingly, by applying an external force to the protruding device formed in the chest tube fixture 320, the chest tube fixture 320 is inserted into the interior of the chest tube 100, and the protruding device is inserted into the fixing hole formed in the chest tube 100, so that the chest tube fixture 320 and chest tube 100 may be fixed. Additionally, the chest tube 100 and the chest tube fixture 320 may be separated by applying an external force to the protruding device formed on the chest tube fixture 320. A plurality of protruding devices may be formed, and the number of protruding devices may be equal to the number of fixing holes. Because of this, the chest tube 100 can be easily inserted into the pleural space by adding the present device to the conventionally employed chest tube 100 and surgical equipment.

The mobile device 2000 may search in real time the in-vivo image information data photographed by the photographing module 200 through a wired or wireless communication network and reproduce the searched in-vivo image information data on a display.

The mobile device 2000 may be configured with a computer such as a desktop personal computer (PC) or a laptop PC, which is a wired or wireless communication device, and may preferably include at least one mobile terminal device selected from a smart phone, smart pad, or smart note. In addition, wired or wireless home appliances and communication devices equipped with a user interface for accessing a cloud server, such as a Palm PC, mobile play-station, digital multimedia broadcasting (DMB) phone with communication function, tablet PC, iPad, can be used comprehensively as the mobile device 2000.

Accordingly, the mobile device 2000 is connected to the photographing module 200 through a wired method, and the in-vivo image information data photographed by the photographing module 200 is searched in real time and reproduced on the display so that the operator can identify the image regardless of location. In another embodiment, the mobile device 2000 may be wirelessly connected to the photographing module 200 to search in real time the in-vivo image information data photographed by the photographing module 200 and reproduce the searched in-vivo image information data on the display.

The drainage container may be formed of two bottles or three bottles, and water may be inserted into the drainage container to adjust the drainage time. Additionally, the drainage container may be connected to a vacuum pump, and when drainage is not smooth, negative pressure may be used to move the drainage substance located inside the pleural space.

The device for inserting the chest tube 100 can facilitate drainage by inserting the chest tube 100 at an appropriate depth by observing the in-vivo image transmitted to the mobile device 2000 through a display formed on the mobile device 2000, and prevent the problem of damaging organs during insertion. In addition, by adding the present device to the conventionally employed chest tube 100, currently employed surgical methods and surgical tools can be used as is.

A method for inserting the chest tube 100 for thoracoscopy using the device for inserting the chest tube 100 for thoracoscopy may comprise the steps of (a) cutting an area to be operated and making a hole in a pleura, (b) combining the photographing module 200, chest tube 100 and handle module 300, (c) photographing the inside of the subject's body by the photographing module 200 when the device for inserting the chest tube 100 is inserted into the subject's pleural space, and then transmitting the photographed in-vivo image to the mobile device 2000 using a wired or wireless communication method, (d) inserting the chest tube 100 based on the in-vivo image reproduced on the mobile device 2000, (e) separating the chest tube 100 and the handle module 300, and (g) connecting the chest tube 100 to the drainage container.

As shown in FIG. 1, in step (a), the axillary line area in the center of the space between the fifth ribs of the human body is incised with a surgical blade, a Mosquito is pushed into the incision area to open the subcutaneous tissue, the muscle tissue is peeled off, and then the resulting exposed pleura is pierced to create an incision window.

After making an incision, Long Kelly and a finger can be used to widen the penetration window formed in the pleura to form a space in advance for the chest tube 100 to enter the pleural space.

After step (a), step (b), which is a step of combining the chest tube 100, photographing module 200, and handle module 300, may be performed. In step (b), the handle fixture 310 having the same diameter as the diameter of the inner surface of the chest tube 100 is screwed together with the handle module 300. Then, the guide part 210 in which the combined length of the photographing part 220 and transmitting part 230 is equal to the length of the chest tube 100 is selected to screw the other side of the guide part 210 with the handle fixture 310, and one side of the handle fixture 310 with the photographing part 220 and transmitting part 230.

After fixing the chest tube fixture 320 to the fixing hole of the chest tube 100, the photographing module 200 coupled with the handle module 300 is inserted in a direction from the other side to one side of the chest tube 100 until the handle fixture 310 contacts the chest tube fixture 320, so that the chest tube 100, photographing module 200, and handle module 300 can be combined.

After step (b) is performed, (c) step in which when the device for inserting the chest tube 100 is inserted into the subject's pleural space, the photographing module 200 photographs the inside of the subject's body, and then transfers the photographed in-vivo image to the mobile device 2000 using a wired or wireless communication method can be performed.

After step (c) is performed, step (d), which is a step of inserting the chest tube 100 based on the in-vivo image reproduced on the mobile device 2000, may be performed. In step (d), the in-vivo image information data transmitted from the photographing module 200 through wired or wireless communication can be reproduced on the display of the mobile device 2000 in real time using the mobile device 2000.

After step (d) is performed, step (e), which is a step of inserting the chest tube 100 based on the in-vivo image reproduced on the mobile device 2000, may be performed. In step (e), the chest tube 100 is inserted based on the in-vivo image reproduced on the mobile device 2000 to insert the chest tube 100 into the appropriate depth, thereby preventing problems of damaging organs.

After step (e) is performed, step (f), which is a step of separating the chest tube 100 and the handle module 300, may be performed. In step (f), after the operator grasps the handle module 300, the handle module 300 can be moved in a direction from one side to the other side of the chest tube 100 to separate the chest tube 100 and the handle module 300.

After step (f) is performed, step (g), which is a step of connecting the chest tube 100 to the drainage tube, may be performed. In step (g), the other side of the chest tube 100 from which the photographing module 200 is removed is connected to the drainage container that stores the drainage substance located in the subject's pleural space, and the substance located inside the pleural space is transferred in a direction from one side to the other side of the chest tube 100 to be drained.

In another embodiment, as shown in FIG. 4, the chest tube 100 may be selectively coupled with a monitoring device 400 equipped with a display, which allows the chest tube 100 to be adjusted by observing the inside of the pleural space with the naked eye. Thus, the chest tube 100 may be introduced into the pleural space.

The guide part 210 may be screwed to the monitoring device 400, and the monitoring device 400 is connected to the upper portion of the chest tube 100, so that when the operator inserts the chest tube 100, he or she can accurately move the chest tube 100 by observing the monitoring part and controlling the chest tube 100 at the same time, instead of the operator looking at an external display and moving his/her gaze back to the chest tube 100 to adjust the chest tube 100.

In another embodiment, a microscope may be inserted inside the chest tube 100, and the microscope may include the monitoring device 400 having an objective lens and a zoom lens, a beam splitter disposed between the zoom lens and the monitoring device 400, a plurality of mirrors, a camera, the transmitting part 230, the lighting device 240, and a power supply.

The zoom lens may be composed of multiple lens modules, and the lenses may be arranged in the following order: objective lens, zoom lens, and monitoring device 400. The photographing part 220 for acquiring a three-dimensional image may be provided between the zoom lens and the eyepiece.

The photographing part 220 may be arranged in the following order: beam splitter, first mirror, second mirror, adjustment device, and camera, and the beam splitter may be placed on the optical path of the microscope. The light from the organs inside the body reflected by the light generated by the lighting device 240 passes through the objective lens, the zoom lens, and then a beam splitter that changes the optical path, and may be divided into the light incident on the monitoring device 400 and the light incident on the photographing part 220.

The light passing through the beam splitter passes through the first and second mirrors that change the optical path, and the light passing through the iris adjuster and focus adjuster placed on the optical path of the first and second mirrors can move to the camera and be incident.

The light incident on the camera may be converted into image data, and the image data may be transmitted to the mobile device 2000 through the transmitting part 230 using a wired or wireless communication method. For this reason, the microscope may be inserted inside the chest tube 100 to observe the inside of the pleural space with the naked eye, thereby inserting the chest tube 100, or the chest tube 100 may be inserted by displaying image data transmitted to the mobile device 2000 using a wired or wireless communication method. Due to this, drainage may be facilitated by inserting the chest tube 100 at an appropriate depth, and problem of damaging organs when inserting the chest tube 100 can be prevented.

By observing the in-vivo image transmitted to the mobile device 2000 using a wired or wireless communication method through the display formed on the mobile device 2000, the chest tube 100 can be inserted at an appropriate depth to facilitate drainage, and problems of damaging organs during insertion can be prevented. In addition, by adding the present device to the conventionally employed chest tube 100, currently employed surgical methods and surgical tools can be used as is.

In another embodiment, as shown in FIGS. 5 and 6, a chest tube fixation module 500 is formed in a cylindrical shape, may include a screw shaft 510 that is provided with a thread on a side thereof, a rotation motor 520 that is coupled to the screw shaft 510 and rotates the screw shaft 510, a moving body 550 that is screwed together with the screw shaft 510, is coupled with a support wing part 540, and moves linearly back and forth to one side and the other side of the screw shaft 510 by rotation of the screw shaft 510, a wing fixing part 530 that is coupled to the rotation motor 520, is in contact with the inner surface of the chest tube, and has a plurality of wings extending in a radial direction, and the support wing part 540 that is coupled to the moving body 550 and wing fixing part 530 and is adjusted to spread or gather the wing fixing part 530 according to the movement of the moving body 550.

One side of the screw shaft 510 is coupled to the rotation motor 520 and may be rotated by the rotation motor 520, and the other side of the screw shaft 510 may be coupled to a screw plate 511 formed in a circular plate shape. The diameter of the screw plate 511 may be larger than the diameter of the moving body 550, so that the moving body 550 may make a linear reciprocating motion along the screw shaft 510 without going out of the screw shaft 510.

The screw thread formed on the side of the screw shaft 510 may be coupled with the screw thread formed on the inner surface of the moving body 550, and as a result, the moving body 550 may be screwed along the screw shaft 510 in one direction and the other direction of the screw shaft 510 by rotation of the screw shaft 510.

The other side of the rotation motor 520 may be coupled to one side of the screw shaft 510, and one side of the rotation motor 520 may be coupled to the wing fixing part 530. A controller controls the rotation of the rotation motor 520 according to the diameter of the chest tube 100 measured by a diameter measurement sensor 560, so that the rotation motor 520 rotates the screw shaft 510 and the moving body 550 may be moved along the screw thread of the screw shaft 510 to one direction and the other direction of the screw shaft 510.

The moving body 550 may include a moving tube 551 that has a thread to be coupled to the thread formed on the screw shaft 510, and a fixing tube 552 that is coupled to the moving tube 551 with a ball bearing. Due to this, the moving tube 551 may be rotated in the rotation direction of the screw shaft 510 by the rotation of the screw shaft 510, and the fixing tube 552 is coupled to the support wing part 540 coupled to the wing fixing part 530, thereby moving the support wing part 540 without rotation.

The wing fixing part 530 may include a fixing plate 531 formed in a circular plate shape and a plurality of stationary wings 532 extending in a radial direction from the edge of the fixing plate 531. The wing fixing part 530 may be formed of a flexible material. The other side of the fixing plate 531 of the wing fixing part 530 may be coupled with one side of the rotation motor 520, and the stationary wings 532 of the wing fixing part 530 may be hingedly coupled to the support wing 542 of the support wing part 540. Additionally, the photographing module 200 may be inserted between the plurality of stationary wings 532, so that one side of the fixing plate 531 and the photographing module 200 may be combined.

The stationary wing 532 may be provided with a wing extension extending outward from the edges of both sides of the stationary wing 532, and a hinge hole formed in the shape of a hole may be formed at the horizontal and vertical centers of the wing extension. One support wing 542 may be located between one wing extension and another wing extension, and one hinge hole and the hinge fixing hole formed in the support wing 542 may be hingedly coupled to another hinge hole.

As a result, even if the angle θ between the support wings 540 changes, the coupling angle formed by the coupling of the support wing 542 and stationary wing 532 changes according to the angle θ between the support wings 540, thereby facilitating the coupling of the wing fixing part 530 and support wing part 540. Due to the wing fixing part 530 formed of a flexible material, when the angle θ between the fixed plate 531 and the stationary wing 532 changes due to an external force, the stationary wing 532 can be moved without being broken.

A plurality of stationary wings 532 may be formed to be spaced apart from the fixing plate 531, and 4 to 10 stationary wings 532 may be formed. Due to the space formed between one stationary wing 532 and the other stationary wing 532, the drainage material inside the chest cavity can be discharged between one stationary wing 532 and the other stationary wing 532.

The drainage substance is discharged into the space formed between one stationary wing 532 and the other stationary wing 532, so that the drainage substance can be drained without separating the photographing module 200 from the chest tube 100.

Due to this, a degree of drainage of the drainage substance may be monitored, and when drainage substance occurs in multiple locations, the pleural space may be monitored with the photographing module 200 and the chest tube 100 may be moved to easily remove the drainage substance. When drainage is completed and the chest tube 100 is removed from the pleural space, problem of damaging organs may be prevented by monitoring the inside of the body using the photographing module 200.

The angle θ between the fixing plate 531 and stationary wing 532 of the wing fixing part 530 may change by the movement of the moving body 550. When the rotation motor 520 rotates clockwise, the screw shaft 510 may rotate clockwise, and the rotation of the screw shaft 510 causes the moving body 550 to move in a direction from one side and the other side of the screw shaft 510. The support wing part 540 coupled to the outer surface of the moving body 550 may move in a direction from one side to the other side of the screw shaft 510 by the movement of the moving body 550, and as a result, the stationary wing 532 of the wing fixing part 530 coupled with the support wing part 540 may be moved toward the other side of the screw shaft 510, thereby increasing the angle θ between the fixing plate 531 and the stationary wing 532.

When the rotation motor 520 rotates counterclockwise, the screw shaft 510 may rotate counterclockwise, and the rotation of the screw shaft 510 causes the moving body 550 to move in a direction from the other side to one side of the screw shaft 510. The support wing part 540 coupled to the outer surface of the moving body 550 may move in a direction from the other side to one side of the screw shaft 510 by the movement of the moving body 550, and as a result, the stationary wing 532 of the wing fixing part 530 coupled with the support wing part 540 may be moved toward one side of the screw shaft 510, thereby reducing the angle θ between the fixing plate 531 and the stationary wing 532.

The stationary wing 532 may be provided with a contact body 533 extending from the upper edge of the stationary wing 532 toward the outer surface, and the contact body may be formed in a round shape. Due to the contact body, even when the angle θ between the fixing plate 531 and the stationary wing 532 changes, the contact area between the stationary wing 532 and the chest tube is formed to be the same, so that the chest tube and the chest tube fixation module 500 can be firmly fixed.

The support wing part 540 may include a support ring 541 that is formed in a shape surrounding the outer surface of the moving body 550, and a plurality of support wings 542 extending in a radial direction from the support ring 541.

The number of support wings 542 may be the same as the number of stationary wings 532, and one stationary wing 532 may be hingedly coupled with one support wing 542. The support wing 542 may be provided with a hinge fixing hole formed in the shape of a hole on the upper side of the support wing 542, and may be hinged with the hinge hole formed on the stationary wing 532.

The support wing part 540 may reciprocate linearly in the direction of one side and the other side of the screw shaft 510 bar by the movement of the moving body 550, so that the distance between the support wing part 540 and the wing fixing part 530 may be changed. When a distance between the support wing part 540 and the wing fixing part 530 increases, the angle θ between the fixing plate 531 and the stationary wing 532 may increase, and when a distance between the support wing part 540 and the wing fixing part 530 is shortened, the angle θ between the fixing plate 531 and the stationary wing 532 may decrease.

The diameter measurement sensor 560 may be coupled to the other side of the screw shaft 510 and, when the diameter measurement sensor 560 is inserted into the chest tube, it may measure the diameter inside the chest tube. According to the inner diameter of the chest tube measured by the diameter measurement sensor 560, the controller controls the rotation motor 520 to change the angle θ between the fixing plate 531 of the wing fixing part 530 and the stationary wing 532.

The chest tube fixation module 500 may be provided with a battery that supplies power to the rotation motor 520, and in another embodiment, the rotation motor 520 may be connected to an outlet by a wire to supply power.

The controller may control transmitting the in-vivo image photographed by the photographing module 200 to the outside using a wireless communication method, and change the angle θ between the fixing plate 531 of the wing fixing part 530 and the stationary wing 532 by controlling the rotation motor 520 according to the diameter of the chest tube measured by the diameter measurement sensor 560.

As shown in FIG. 7, the chest tube fixation module 500 may change the angle θ between the fixing plate 531 of the wing fixing part 530 and the stationary wing 532 according to the diameter of the chest tube to contact the inside of the chest tube, so that the chest tube fixation module 500 and chest tube may be fixed.

The angle θ between the fixing plate 531 of the wing fixing part 530 and the stationary wing 532 may change by the movement of the moving body 550. When the rotation motor 520 rotates clockwise, the screw shaft 510 may rotate clockwise, and the rotation of the screw shaft 510 causes the moving body 550 to move in a direction from one side to the other side of the screw shaft 510. The support wing part 540 coupled to the outer surface of the moving body 550 may move in a direction from one side to the other side of the screw shaft 510 by the movement of the moving body 550, and as a result, the stationary wing 532 of the fixing wing part 530 coupled with the support wing part 540 may be moved toward the other side of the screw shaft 510, thereby increasing the angle θ between the fixed plate 531 and the stationary wing 532. Because of this, the chest tube fixation module 500 can be easily fixed inside the chest tube with a large diameter.

When the rotation motor 520 rotates counterclockwise, the screw shaft 510 may rotate counterclockwise, and the rotation of the screw shaft 510 causes the moving body 550 to move in a direction from the other side to one side of the screw shaft 510. The support wing part 540 coupled to the outer surface of the moving body 550 may move in a direction from the other side to one side of the screw shaft 510 by the movement of the moving body 550, and as a result, the stationary wing 532 of the wing fixing part 530 coupled with the support wing part 540 may be moved toward one side of the screw shaft 510, thereby reducing the angle θ between the fixing plate 531 and the stationary wing 532. Because of this, the chest tube fixation module 500 can be easily fixed inside the chest tube with a small diameter.

When the inner diameter (D) of the chest tube is large in the order of D1, D2, and D3, the angle (θ), which is an angle between the fixing plate 531 and the stationary wing 532, may be formed to be large in the order of θ₁, θ₂, θ₃.

In another embodiment, as shown in FIG. 10, the camera fixation module 600 may include a camera cover 610 that is in contact with the photographing module 200 and is formed in a shape surrounding the photographing module 200, and a pin part 620 that is provided with a plurality of pins formed in an upward direction from the outer surface of the camera cover 610 and is in contact with the chest tube 100.

The camera cover 610 is formed in a shape that surrounds the photographing module 200. The material of the camera cover 610 may be formed of synthetic resin, rubber, and silicone. As a result, the camera cover 610 is stretched by an external force, so that the photographing module 200 may be easily inserted into the camera cover 610.

The pin part 620 includes a plurality of pins formed in an upward direction from the outer surface of the camera cover 610, and the plurality of pins may be in contact with the inner surface of the chest tube 100.

The camera fixation module 600 may be inserted from the other side of the chest tube 100 coupled with the drainage container to one side of the chest tube 100 entering the plural space, and the camera fixation module 600 may be formed to be inclined to form an acute angle with the flow direction of the fluid flowing from one side of the chest tube 100 to the other side of chest tube 100.

Because of this, even if the flow amount of fluid is large or negative pressure is applied to the chest tube 100, the plurality of pins forms an acute angle with the flow direction of the fluid and is fixed to the inside of the chest tube so that the camera fixation module 600 is not affected by the flow or negative pressure, and may be easily fixed to the inner surface of the chest tube 100.

In the plurality of pins, a pin angle α forming an acute angle may be formed between a tangent line parallel to the central axis of the camera cover 610 while contacting the plurality of pins on the outer peripheral surface of the camera cover 610 and the plurality of pins.

As shown in FIG. 11, the pin angle (α) may be changed by applying an external force to the plurality of pins, and the inner surface of the chest tube 100 and the plurality of pins with the modified pin angle (α) are in contact. Thus, the plurality of pins and the inner surface of the chest tube 100 may be fixed.

When the inner diameter of the chest tube 100 is a first diameter D1, an external force may be applied to the plurality of pins to change the pin angle α and the first pin angle α₁ may be formed. When the inner diameter of the chest tube 100 is a second diameter (D2), an external force may be applied to the plurality of pins to change the pin angle α and the second pin angle α₂ may be formed. When the inner diameter of the chest tube 100 is a third diameter (D3), an external force may be applied to the plurality of pins to change the pin angle α and the third pin angle α₃ may be formed.

When the inner diameter (D) of the chest tube 100 is large in the order of D1, D2, and D3, the pin angle α forming an acute angle between a tangent line parallel to the central axis of the camera cover 610 while contacting the plurality of pins on the outer peripheral surface of the camera cover 610 and the plurality of pins may be formed to be large in the order of α₁, α₂, and α₃. In this case, the length of the pin may be longer than the radius of the inner surface of the chest tube 100.

Due to this, regardless of the diameter of the chest tube 100, the camera fixation module 600 may be inserted into the chest tube 100 by applying an external force to the plurality of pins according to the diameter of the chest tube 100. Regardless of the diameter of the chest tube 100, the camera fixation module 600 and the inner surface of the chest tube 100 may be fixed to easily change the chest tube 100 according to the amount of drainage.

The pin part 620 may be formed in plural number on the outer surface of the camera cover 610 parallel to the central axis of the camera cover 610, and fluid may flow along the space formed by the camera cover 610 and the plurality of pin parts 620.

That is, fluid may flow along one space formed by one pin part 620, the other pin part 620, the camera cover 610, and the chest tube 100, allowing smooth drainage. Four to six pin parts 620 may be formed, and four pin parts 620 may be formed in the disclosure, but the number is not limited thereto.

A (S1) zone formed by the first pin part 620 and the second pin part 620, (S2) zone formed by the second pin part 620 and the third pin part 620, and (S3) zone formed by the third pin part 620 and the fourth pin part, and (S4) zone formed by the fourth pin part 620 and the first pin part 620 may be formed.

Fluid may flow through the space formed in the (S1, 2, 3, 4) zones. Accordingly, through the space formed in the (S1, 2, 3, 4) zones in which the photographing module 200 is inserted into the chest tube 100 using the camera fixation module 600, the fluid is drained, and the drainage may be performed without separating the photographing module 200 from the chest tube 100.

In addition, the pin part 620 may be provided in plural numbers, and fluid flows along a space formed by one pin, another pin, the camera cover 610, and the chest tube 100 to facilitate smooth drainage. 3 to 5 pins may be formed in one pin part 620, and in the disclosure, 3 pins may be formed, but the number is not limited thereto.

A (S5) zone formed by the first and second pins and (S6) zone formed by the second and third pins may be formed. The fluid flowing through the space formed in the (S1, 2, 3, 4) zones also flows the space formed in the (S5, 6) zones. Thus, the liquid formed in the pleural space is drained from one side of the chest tube 100 to the other side of the chest tube 100, and drainage may be easily performed without separating the photographing module 200 from the chest tube 100.

Due to this, drainage may be performed while fixing the photographing module 200 inside the chest tube 100 without removing it. When drainage substance occurs in multiple locations, the chest tube is monitored with the photographing module 200 and drainage is complete on a portion formed in the pleural space. Then, the inside of the pleural space is monitored through the photographing module 200, the chest tube 100 is moved, and drainage is performed on other portion formed in the pleural space, so that the drainage substance can be easily removed.

In addition, it is possible to monitor the amount and degree of drainage of objects located in the chest tube, so the completion point of drainage can be predicted. Also, when an object is removed from the pleural space, the internal of the body can be monitored using the photographing module 200 to prevent damage to organs, and the surgical site can be quickly sutured.

A method for inserting the chest tube 100 for thoracoscopy using the device for inserting the chest tube 100 for thoracoscopy may comprise the steps of (a) cutting an area to be operated and making a hole in a pleura, (b) combining the photographing module 200 and camera fixation module 600 and inserting the photographing module 200 and camera fixation module 600 in the pleura space, (c) photographing the inside of the subject's body by the photographing module 200 when the device for inserting the chest tube 100 is inserted into the subject's pleural space, and then transmitting the photographed in-vivo image to the mobile device 2000 using a wired or wireless communication method, (d) inserting the chest tube 100 based on the in-vivo image reproduced on the mobile device 2000, (e) separating the chest tube 100 and the handle module 300, and (g) connecting the chest tube 100 to the drainage container.

As shown in FIG. 1, in step (a), the axillary line area in the center of the space between the fifth ribs of the human body is incised with a surgical blade, a Mosquito is pushed into the incision area to open the subcutaneous tissue, the muscle tissue is peeled off, and then the resulting exposed pleura is pierced to create an incision window.

After making an incision, Long Kelly and a finger can be used to widen the penetration window formed in the pleura to form a space in advance for the chest tube 100 to enter the pleural space.

After step (a), step (b), which is a step of combining the photographing module 200 and camera fixation module 600 and the photographing module 200 and camera fixation module 600 are inserted in the pleural space, may be performed. In step (b), as shown in FIG. 9, the camera cover 610 may be inserted into the photographing module 200.

When inserting the camera fixation module 600 into the chest tube 100, an external force is applied to the plurality of pin parts 620 according to the diameter of the chest tube 100 to change the pin angle α to insert the camera fixation module 600 inside the chest tube 100.

The camera fixation module 600 may be formed inclined to form an acute angle with the flow direction of the fluid flowing from one side of the chest tube 100 to the other side of the chest tube 100. Thus, even if the flow amount of fluid is large or negative pressure is applied to the chest tube 100, the plurality of pins form an acute angle with the flow direction of the fluid and are fixed to the inside of the chest tube so that the camera fixation module 600 is not swept away by the flow and negative pressure and may be easily fixed to the inner surface of the chest tube 100, thereby preventing the chest tube fixation module 500 from being sucked into the drainage container by the flow.

After step (b) is performed, step (c) in which when the device for inserting the chest tube 100 is inserted into the subject's pleural space, the photographing module 200 photographs the inside of the subject's body, and then transfers the photographed in-vivo image to the mobile device 2000 using a wired or wireless communication method may be performed.

After step (c) is performed, step (d), which is a step of inserting the chest tube 100 based on the in-vivo image reproduced on the mobile device 2000, may be performed. In step (d), the in-vivo image information data transmitted from the photographing module 200 using the mobile device through a wired or wireless communication method may be reproduced on the display of the mobile device 2000 in real time.

After step (d) is performed, step (e), which is a step of inserting the chest tube 100 based on the in-vivo image reproduced on the mobile device 2000, may be performed. In step (e), the chest tube 100 is inserted based on the in-vivo image reproduced on the mobile device 2000 to insert the chest tube 100 into the appropriate depth of the chest tube, thereby preventing problems of damaging organs.

After step (e) is performed, step (e), which is a step of connecting the chest tube 100 to the drainage container, may be performed. In step (e), the other side of the chest tube 100 from which the photographing module 200 is removed is connected to the drainage container that stores the drainage substance located in the subject's pleural space, and the substance located inside the pleural space is transferred and drained from one side to the other side of the chest tube 100.

In addition, a degree of drainage of the drainage substance may be monitored by draining the drainage substance without separating the photographing module 200 from the chest tube 100. When the drainage substance occurs in multiple locations, the chest tube is monitored with the photographing module 200. The drainage substance may be easily removed by moving the chest tube 100, and when drainage is completed and the chest tube 100 is removed from the pleural space, it is possible to prevent organ damage by monitoring the inside of the body through the photographing module 200.

With the chest tube 100 inserted into the chest tube by the operator's breathing, the drainage substance may move from one side to the other side of the chest tube 100 and enter the inside of the drainage container. The drainage container may be formed of two bottles or three bottles, and water may be inserted into the drainage container to adjust the drainage time. Additionally, the drainage container may be connected to a vacuum pump, and when drainage is not smooth, negative pressure may be used to move the drainage substance located inside the pleural space.

The above-mentioned embodiments of the disclosure are merely examples, and it will be understood by those skilled in the art that various modifications may be made without departing from the technical spirit and scope or essential features of the disclosure. Therefore, it should be understood that the embodiments described above are for purposes of illustration only in all aspects and are not intended to limit the scope of the disclosure. For example, each component described in a single form may be implemented in a distributed form, and similarly, components described in the distributed form may be implemented in a combined form.

The scope of the disclosure is defined by the appended claims, and it should be construed that all modifications or variations derived from the meaning, scope, and equivalent concept of the claims fall within the scope of the disclosure.

### DESCRIPTION OF REFERENCE NUMERALS

100: chest tube
200: photographing module
210: guide part
220: photographing part
230: transmitting part
240: lighting device
300: handle module
310: handle fixture
320: chest tube fixture
400: monitoring device
500: chest tube fixation module
510: screw shaft
511: screw plate
520: rotation motor
530: wing fixing part
531: fixing plate
532: stationary wing
533: contact body
540: support wing part
541: support ring
542: support wing
550: moving body
551: moving tube
552: fixing tube
560: diameter measurement sensor
600: camera fixation module
610: camera cover
620: pin part
2000: mobile device

1. A device for inserting a chest tube for thoracoscopy, comprising:
a photographing module configured to photograph an inside of a subject's body and transmit a photographed in-vivo image to an outside;
a chest tube having a rigid tube shape, wherein photographing module is inserted and installed in the chest tube;
a guide part coupled to the photographing module and configured to guide the photographing module into the chest tube;
a handle module coupled to the guide part and configured to move the guide part and photographing module; and
a mobile device configured to receive data from the photographing module, and to search for and reproduce the in-vivo image photographed by the photographing module in real time,
wherein when the chest tube coupled with the photographing module is inserted into a pleural space, the photographing module photographs an inside of the pleural space and transmits the photographed image to the mobile device through a wired or wireless communication method, so that an operator monitors the inside of the pleural space regardless of location.

2. The device of claim 1, wherein the photographing module and guide part are selectively separated from the chest tube.

3. The device of claim 1, wherein the photographing module includes:
a photographing part configured to photograph the inside of the subject's body; and
a transmitting part coupled to the photographing part and configured to receive the in-vivo image transmitted from the photographing part to transmit the in-vivo image to the mobile device through the wired or wireless communication method.

4. The device of claim 3, wherein the photographing part includes:
a camera configured to photograph the inside of the subject's body; and
a lighting device coupled with the camera, formed in plural number, and configured to emit light to illuminate the inside of the body.

5. The device of claim 1, wherein the photographing module includes:
a camera cover that is in contact with the photographing module and is formed in a shape surrounding the photographing module; and
a pin part that is provided with a plurality of pins formed in an upward direction from an outer surface of the camera cover and is in contact with the chest tube.

6. The device of claim 5, wherein a material of the camera cover is formed of synthetic resin, rubber, and silicone.

7. The device of claim 5, wherein the plurality of pins is inclined to form an acute angle with a flow direction of fluid flowing in one direction.

8. The device of claim 7, wherein in the plurality of pins, a pin angle α forming an acute angle is formed between a tangent line parallel to a central axis of the camera cover while contacting the plurality of pins on an outer peripheral surface of the camera cover and the plurality of pins.

9. The device of claim 8, wherein when an external force is applied to the plurality of pins, the pin angle is changed by the external force, so that an inner surface of the chest tube comes into contact with the plurality of pins, and the plurality of pins and the chest tube are fixed.

10. The device of claim 7, wherein the pin part is formed in plural number, and fluid flows along a space formed by the camera cover and the plurality of pin parts.

11. A method for inserting a chest tube for thoracoscopy using the device for inserting the chest tube for thoracoscopy of claim, the method comprising:
(a) cutting an area to be operated and making a hole in a pleura;
(b) inserting the photographing module into the pleural space;
(c) photographing the inside of the subject's body by the photographing module when the device for inserting the chest tube is inserted into the subject's pleural space, and then transmitting the photographed in-vivo image to the mobile device using the wired or wireless communication method;
(d) inserting the chest tube based on the in-vivo image reproduced on the mobile device; and
(e) connecting the chest tube to a drainage container.

12. The method of claim 11, wherein the drainage container stores drainage substance located in the subject's pleural space.
